(19) United States
(12) Patent Application Publication
(10) Pub. No.: US 2018/0348215 A1
(43) Pub. Date: Dec. 6, 2018

(54) SYSTEMS AND METHODS FOR MEASURING A CONCENTRATION OF AN ANALYTE

(71) Applicant: Waters Technologies Corporation, Milford, MA (US)

(72) Inventors: Lingyun Chen, Scarborough, ME (US); Jianmin Liu, Framingham, MA (US)

(21) Appl. No.: 15/992,427

(22) Filed: May 30, 2018

Related U.S. Application Data
(60) Provisional application No. 62/512,897, filed on May 31, 2017.

(51) Int. Cl. G01N 33/558; G01N 33/543

(52) U.S. Cl. CPC G01N 33/558; G01N 33/54346

(57) ABSTRACT

The present disclosure relates to systems and methods for determination of analyte concentrations in solution using materials that are pre-dispensed and dried on conjugate pads or other solid substrates. The solution including the analyte can reconstitute the dried material within a vial to produce a specified concentration of material in the vial. The resulting solution can be used in lateral flow testing.

### TECHNICAL FIELD

The present disclosure relates generally to systems, methods and kits for measuring a concentration of an analyte, such as, for example a toxin. In particular, the present disclosure relates to systems, methods, and kits which employ a dry probe material that can be easily and effectively reconstituted to provide a specific concentration of probe material in solution for use in a lateral flow test.

### BACKGROUND

Lateral flow testing is used to assess concentrations of an analyte in solution. In the realm of food safety, lateral flow testing can be used to test for the presence of toxins such as mycotoxins that can naturally occur on food products destined for animal or human consumption. The robustness of lateral flow testing devices allows for more active testing of products at the source such as at farms or food-preparation facilities.

### SUMMARY

The present disclosure relates to systems, methods, and kits for determination of analyte concentrations in solution using materials that are pre-dispensed and dried on conjugate pads or other solid substrates.

In one aspect, the present disclosure relates to a system for measuring the concentration of an analyte. The system includes a vial having an open end. The system also includes a conjugate pad including dry probe material. The conjugate pad is sized to be disposed within the vial. The conjugate pad includes an amount of dry probe material such that placing the conjugate pad in contact with a liquid sample including the analyte within the vial reconstitutes the dry probe material to provide a specified concentration of probe material in solution.

In another aspect, the present disclosure relates to a method of measuring the concentration of an analyte. The method includes placing a liquid sample including the analyte into a vial having an open end and including a conjugate pad. The conjugate pad includes dry probe material. The method further includes agitating the liquid in the vial to reconstitute the dry probe material to provide a specified concentration of probe material in solution. The method also includes contacting a lateral flow test strip of a lateral flow device with the liquid sample in the vial. The method also includes analyzing an indicator on the lateral flow device.

In another aspect, the present disclosure relates to a method of producing an analyte measurement system. The method includes dispensing a specified amount of probe material onto a conjugate pad. The method also includes drying the conjugate pad to produce a conjugate pad including dry probe material. The method further includes placing the conjugate pad into a vial having an open end.

In yet another aspect, the present disclosure relates to a method of producing a sample in solution. The method includes dispensing a specified amount of the sample onto a conjugate pad. The method also includes drying the conjugate pad to produce a conjugate pad including dry sample. The method further includes placing the conjugate pad into a vial having an open end. The method also includes placing a liquid into the vial to reconstitute a specified concentration of sample in solution in the vial.

In yet another embodiment, the present disclosure relates to a kit. The kit includes a system for measuring the concentration of an analyte. The system includes a vial having an open end and a conjugate pad including dry probe material and sized to be disposed within the vial. The conjugate pad includes an amount of dry probe material such that placing the conjugate pad in contact with a liquid sample including the analyte within the vial reconstitutes the dry probe material to provide a specified concentration of probe material in solution. The system also includes a pipette. The pipette includes one or more fill indicator lines to indicate volumes of fluid for reconstituting the dry probe material to a specified concentration. The kit further includes instructions for drawing a liquid sample including the analyte into the pipette to one of the one or more fill indicator lines. The kit further includes instructions to dispense the liquid sample into the vial. The kit further includes instructions to agitate the liquid in the vial to reconstitute the dry probe material. The kit further includes instructions to contact a lateral flow test strip of a lateral flow device with the liquid sample in the vial.

Embodiments of the above aspects can include one or more of the following features. In some embodiments, the dry probe material includes conjugated metal nanoparticles or polymer nanoparticles. In some embodiments, a length and a width of the conjugate pad are each in a range from 2 mm to 20 mm. In some embodiments, the systems further comprise a lateral flow device wherein contacting a lateral flow test strip of the lateral flow device with the liquid sample in the vial causes an indicator to appear on the lateral flow device. In some embodiments, the vial includes a volume indicator mark to indicate a volume of the liquid sample to be placed therein. In some embodiments, the amount of probe material dispensed on the conjugate pad is in a range of 0.01 to 2 optical density measured at maximum absorption wavelength when reconstituted in the liquid sample.

The systems, methods and kits of the present disclosure provide several advantages over the prior art. For example, the systems, methods, and kits of the present disclosure can provide a low cost, easy to use system for producing a specified concentration of a small amount of a particular solution from a dried probe material. By drying the probe material on a conjugate pad, systems, methods, and kits of the present disclosure provide a stable way to store the probe material that also allows for rapid and thorough reconstitution. Systems, methods, and kits of the present disclosure can produce better response or sensitivity than conventional methods.

### BRIEF DESCRIPTION OF THE DRAWINGS

The invention will be more fully understood from the following detailed description taken in conjunction with the accompanying drawings, in which:

FIGS. 1A and 1B illustrate components of a system for measuring the concentration of an analyte in accordance with various embodiments described herein;

FIG. 2 illustrates a kit for measuring the concentration of an analyte in accordance with various embodiments described herein;

FIG. 3 illustrates a method of measuring a concentration of an analyte in accordance with various embodiments described herein;

FIG. 4 illustrates a method of producing an analyte measurement system in accordance with various embodiments described herein; and

FIG. 5 illustrates a method of producing a sample in solution in accordance with various embodiments described herein.

### DETAILED DESCRIPTION

Systems and methods described herein provide an economical way to measure the concentration of an analyte in solution. For example, the systems and methods described herein utilize a conjugate pad including a dry probe material located within a vial. The amount of dry probe material on the pad is chosen to provide a specified concentration of probe material in solution once reconstituted. By using a conjugate pad including a dry probe material as described herein, reconstitution of the probe material occurs quickly and the yield of probe material in solution is accurate. In addition, producing the conjugate pad including a dry probe material is cost-effective and rapid.

As used herein, the term “optical density” or “OD” is the logarithm of the ratio of light intensity transmitted through a standard thickness of sample to the light intensity incident on the sample. Optical density is greater where the concentration of analyte in the sample is greater. In some embodiments described herein, the optical density is measured at the wavelength for which maximum absorption occurs when the probe material is in solution.

As used herein, a “probe material” is material that can interact with the analyte to cause a measurable change in properties of the system. For example, some embodiments of the present disclosure use a probe material that can selectively target and bind to a region of the analyte (such as an antibody/antigen system) to form a compound system. In other embodiments, the probe material can chemically react with the analyte to produce a compound having a detectably different chemical structure. In some embodiments, the probe material can include, for example, a visible beacon or can induce a color change in solution.

As used herein, the term “about” means that the numerical value is approximate and small variations would not significantly affect the practice of the disclosed embodiments. Where a numerical limitation is used, unless indicated otherwise by the context, “about” means the numerical value can vary by ±10% and remain within the scope of the disclosed embodiments.

In known systems, colloidal gold nanoparticles conjugated to an antibody are reacted with an analyte in solution to determine a concentration of the analyte. In these systems, the conjugated gold nanoparticles are often provided in a dry format to provide extended shelf-life and reduce weight for shipping. To dry the conjugated nanoparticles or other probe material without sacrificing potency, the probe material is often lyophilized or air dried directly in the vial (with or without centrifugation to concentrate the sample). Lyophilization is a costly and specialized process, and providers of lyophilization as a service often utilize only 96-well microtiter plates that are difficult to manipulate by a human operator and wasteful in situations when only a single test is needed. Air-dried samples often do not fully reconstitute in solution because some of the material becomes adsorbed on the surface of the vial. As a result, the final concentration of probe material in solution is highly variable. Systems and methods described herein provide a low-cost and reliable way to reconstitute a probe material to a prescribed concentration within a vial for interaction with an analyte.

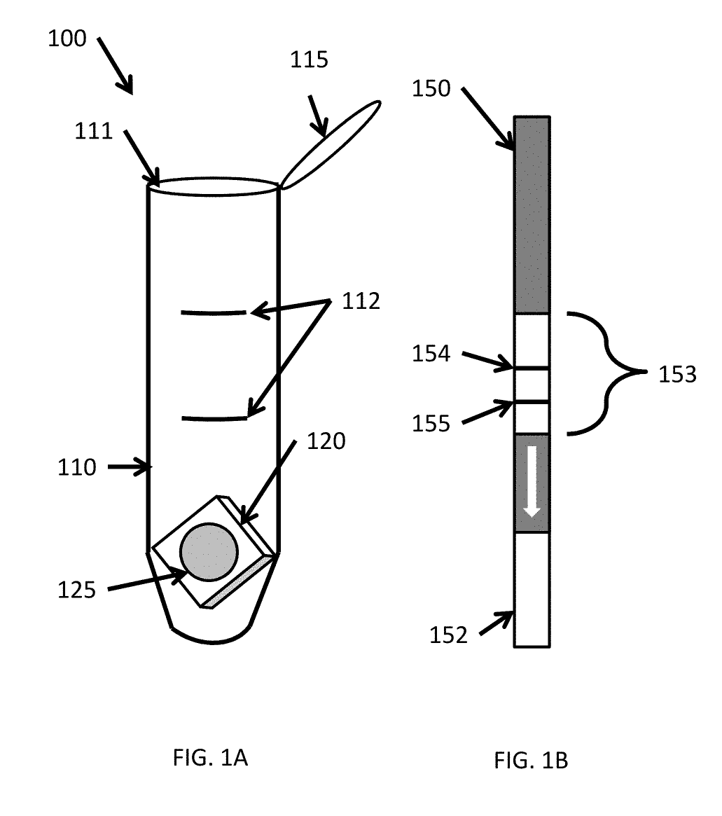

FIGS. 1A and 1B illustrate components of a system 100 for measuring the concentration of an analyte according to various embodiments described herein. The system 100 can include a vial 110 having an open end 111 with a conjugate pad 120 disposed therein. The conjugate pad 120 can include dry probe material 125. Dispensing a liquid sample into the vial 110 can reconstitute the dry probe material to provide a specified concentration of probe material in solution.

The vial 110 can be made of a variety of materials including plastic or glass materials. In some embodiments, the vial 110 can include polycarbonate. In some embodiments, the vial 110 is made of a non-reactive material that is resistant to interaction with materials placed therein. In some embodiments, the vial 110 can include a cap 115 that seals the open end 111. The vial 110 can include one or more volume indicator marks 112 to indicate to a user the appropriate fill volume of the liquid sample to achieve a desired concentration of probe material in solution. In some embodiments, the volume indicator marks 112 can be printed onto the exterior of the vial 110 or can be molded into a surface of the vial 110 (for example, as raised portions or depressions on the surface). In some embodiments, the volume of the vial can be in a range from 200 μL to 3 mL. In a preferred embodiment, the volume of the vial is 1.5 mL.

In some embodiments, the capped vial 110 can be stored within a moisture-proof plastic bag including a desiccant to further ensure the dry probe material is maintained in a low-moisture environment.

The conjugate pad 120 can be sized to be disposed within the vial 110. In some embodiments, the length or width of the conjugate pad can be in a range from 2 mm to 20 mm. In some embodiments, the conjugate pad 120 can be small enough to reliably release the dry probe material into the solution while minimizing agitation time. The conjugate pad 120 can be made from a variety of materials. For example, the conjugate pad 120 can include glass fiber, polyester, porous plastics such as those from POREX® (Fairburn, Ga.), or any other appropriate material. In some embodiments, the conjugate pad 120 can be a portion of a roll of conjugate pad material. That is, in some embodiments, the conjugate pad 120 can be provided separately from the vial 110 and in addition be sized at a place of use by cutting or detaching a piece from a roll or strip of conjugate pad material.

In some embodiments, the conjugate pad 120 can have a high porosity or high surface-to-volume ratio. The high surface-to-volume ratio can advantageously allow the probe material to spread within the conjugate pad 120 upon application and dry quickly. In addition, the high porosity can allow the reconstituting solution to wash through the entire conjugate pad 120 rapidly and enable fast and thorough release of the dry probe material into solution. In some embodiments, the conjugate pad 120 releases greater than 70%, 80%, 90%, 95%, or 99% of the dry probe material by weight. In some embodiments, the high efficiency of release of dry probe material 125 from the conjugate pad 120 can enable better response of the system during lateral flow tests than conventional systems.

The probe material can be dispensed onto the conjugate pad 120 and dried in a variety of ways. For example, the conjugate pad 120 can be formed on a roll and dispensed in a straight line. As the roll of conjugate pad 120 is unspooled, the liquid sample including the probe material can be dispensed at a controlled rate onto the unspooling roll using a liquid dispensing system. In some embodiments the controlled rate can be changed or updated to produce a specified amount per volume or application area (i.e., concentration of liquid sample). Downstream of the liquid dispensing system, the conjugate pad 120 can be dried using forced air (e.g., hot or heated air/gas) or direct or radiant heaters or can be dried at room temperature using ambient air. The roll of conjugate pad can then be cut into appropriate sized pieces that can be disposed within the vial 110. In some embodiments, the probe material can be sprayed onto the conjugate pad 120. For example, the probe material can be atomized into small droplets that are sprayed at a controlled rate onto the conjugate pad 120 as the conjugate pad 120 is moved past a sprayer.

In some embodiments, the optical density of the probe material deposited onto the conjugate pad 120 can be in a range from 1 to 50. In some embodiments, the amount of probe material dispensed on the conjugate pad is such that the reconstituted probe material in a liquid sample has an optical density in a range from 0.01 to 2 measured at maximum absorption wavelength.

The dry probe material 125 can include a variety of probes that can interact with the analyte in a variety of ways. In some embodiments, the dry probe material 125 can include conjugated or bare metal nanoparticles. In some embodiments, the metal nanoparticles can be gold nanoparticles or silver nanoparticles. In some embodiments, the metal nanoparticles can have bare diameters in a range from 20 to 80 nm. In some embodiments, the dry probe material can include non-metal particles such as polymer nano- or microparticles, chitosan nanoparticles, or carbon nanostructures. Polymer nanoparticles can include silica or polystyrene and can range in diameter from 20 nm to 1 μm. In some embodiments, the dry probe material 125 can include a fluorescent or luminescent substance.

In some embodiments, the dry probe material 125 remains monodisperse when dried. In other words, the reconstituted probe material can have about the same level of dispersity as the original sample before it was dispensed and dried on the conjugate pad 120. In some embodiments, the dry probe material 125 can have the same chemical or biological reactivity when reconstituted as the original sample had before it was dispensed and dried on the conjugate pad 120. The dry probe material can have an extended shelf-life as compared to probe material in solution.

In some embodiments, the dry probe material 125 can have a targeting moiety that binds to the analyte. For example, the dry probe material 125 can include gold nanoparticles conjugated covalently or passively to antibodies, proteins, deoxyribonucleic acid (DNA), oligonucleotides, or any other suitable targeting element. In some embodiments, the targeting moiety can bind to mycotoxins or metabolic products created therefrom including, but not limited to, aflatoxins, citrinins, deoxynivalenols (vomitoxins), fumonisins, ochratoxins, zearalenones, T-2, and HT-2.

In some embodiments, the dry probe material 125 can be reconstituted directly in the liquid test sample including the analyte. In alternative embodiments, the dry probe material 125 can be reconstituted in a precursor solution such as water or a buffered solution. The liquid test solution including the analyte can then be dispensed into the vial including the reconstituted probe material. In various embodiments, the vial 110 including the conjugate pad 120 can be agitated to promote reconstitution of the dry probe material in solution. For example, the vial 110 can be shaken, stirred, or vortexed. In various embodiments, the agitation can last for 15, 30, 45, 60, 120, or 300 seconds.

In some embodiments, the system 100 can further include a lateral flow device 150 (shown in FIG. 1B) having a lateral flow test strip 152. The lateral flow test strip 152 can be placed contact with the liquid sample in the vial to quantitatively determine the concentration of an analyte in the sample. For example, the lateral flow test strip 152 can draw the solution into the lateral flow device 150 and to an indicator region 153. In some embodiments, the indicator region 153 can be configured to display a control indicator line 154 and a test indicator line 155. In accordance with various embodiments, the control indicator line 154 of the lateral flow device 150 can be configured to appear at the conclusion of any successful test. In other words, the absence of the control indicator line 154 can indicate that the test has failed. Test failure can occur, for example, due to improper preparation of the sample or probe material. In accordance with various embodiments, the degree to which the test indicator line 154 appears at the conclusion of a successful test can correlate to the concentration of the analyte in solution. In some embodiments, a test indicator line 154 that appears at the same intensity as the control indicator line 154 can indicate that the analyte is not present in the sample. In other words, the test indicator line 154 is absent when the concentration of the analyte is at or above a testing limit. Intensity values for the test indicator line 154 between these extremes can be proportionate to the concentration value of the analyte in solution.

In some embodiments, probe material reconstituted from systems described herein can have a better response or sensitivity than that produced by conventional methods. To illustrate some of the advantages of the technology of the present disclosure, the following comparative experiment was conducted.

In this comparative example, performance of a system in accordance with the present disclosure (the “test” system) was compared to the performance of a conventional system. In the test system, a conjugate pad including dry probe material was prepared and placed in a vial. The liquid sample was added to the vial, and the probe material was reconstituted. A lateral flow device was placed into the sample/probe solution, and the resulting indicators produced on the lateral flow device were analyzed. In the conventional system, a lateral flow device including probe material was placed into the liquid sample, and the resulting indicators on the lateral flow device were analyzed.

Samples of conjugated gold nanoparticles were prepared at a concentration of 32 OD measured at 540 nm for use in the test system. Active gold particles were prepared by conjugating to M₁ antibody targeted to aflatoxin M₁.

To prepare the conjugate pad including dry probe material, 2 microliters of a conjugated gold nanoparticle solution was dispensed onto a glass fiber pad (Ahlstrom 8951, commercially available from Ahlstrom-Munksjo Corporation, Stockholm, Sweden). The solution was dispensed linearly onto the pad at a rate of 2 microliters/cm at 3 PSI. The conjugate pad was dried at 50° C. for six minutes. A 6 mm by 4 mm portion of the conjugate pad was removed and placed into a 1.5 mL vial.

To prepare a conventional system, conjugated gold particles were prepared as described above at a concentration of 15 OD. A quantity of 1.5 microliters/cm of the particle solution was dispensed onto a glass fiber conjugate pad. A 6 mm by 4 mm portion of the conjugate pad was removed and assembled directly onto the lateral flow test strip.

For the test system, the lateral flow test strip was assembled as follows. A blank conjugate pad was created that includes M₁ blocker. The M₁ blocker was dispensed linearly onto the blank conjugate pad at a rate of 10 microliters/cm. The blank conjugate pad, nitrocellulose membrane (UniSart® CN95 membrane, Sartorius GmbH, Gottingen, Germany), and sample pad were then laminated onto a backing pad coated with adhesive. The complete laminated pad was then cut into 4 mm strips to form the lateral flow test strips. To prepare lateral flow test strips for the conventional system, the procedure described above for the test system was followed substituting the gold nanoparticle conjugate pad for the blank conjugate pad.

Positive and negative samples were created to test the relative response of the test system and the conventional system during a lateral flow test. The negative sample was aflatoxin M₁-free whole milk while the positive sample was whole milk spiked with aflatoxin M₁ at a concentration of 50 parts per trillion (ppt). As described above, use of the conventional system involved placing the lateral flow device test strip into contact with the liquid sample. The sample was then incubated at 40° C. for ten minutes, and the lateral flow device was analyzed thereafter.

To employ the test system, the dried probe material must first be reconstituted. To reconstitute the dried probe material on the conjugate pad in the vial, 200 microliters of a liquid sample was added to the vial including the conjugate pad having dry probe material. The vial was vortexed at high speed for fifteen seconds. The lateral flow device test strip was then placed into contact with the liquid in the vial, and the vial was placed on an incubator at 40° C. for ten minutes. The lateral flow device was analyzed thereafter.

The ratio of the light absorption value for the test line to the value for the control line can be used to quantitatively measure the response of the system. Table 1 below shows the test/control (T/C) ratio values for each test condition wherein each test condition was repeated three times:

TABLE 1

| | Test System | Conventional system |
|---|---|---|
| | (T/C ratio) | (T/C ratio) |
| 0 ppt aflatoxin M₁ | 2.4, 2.3, 2.4 | 2.4, 2.5, 2.3 |
| (negative sample) | | |
| 50 ppt aflatoxin M₁ | 1.5, 1.6, 1.5 | 1.9, 2.0, 1.8 |
| (positive sample) | | |

As shown in Table 1, embodiments of the present application that reconstitute probe material from a conjugate pad including dried probe material provide better response (i.e., the difference of the T/C ratio values between negative sample and positive sample) over the conventional test

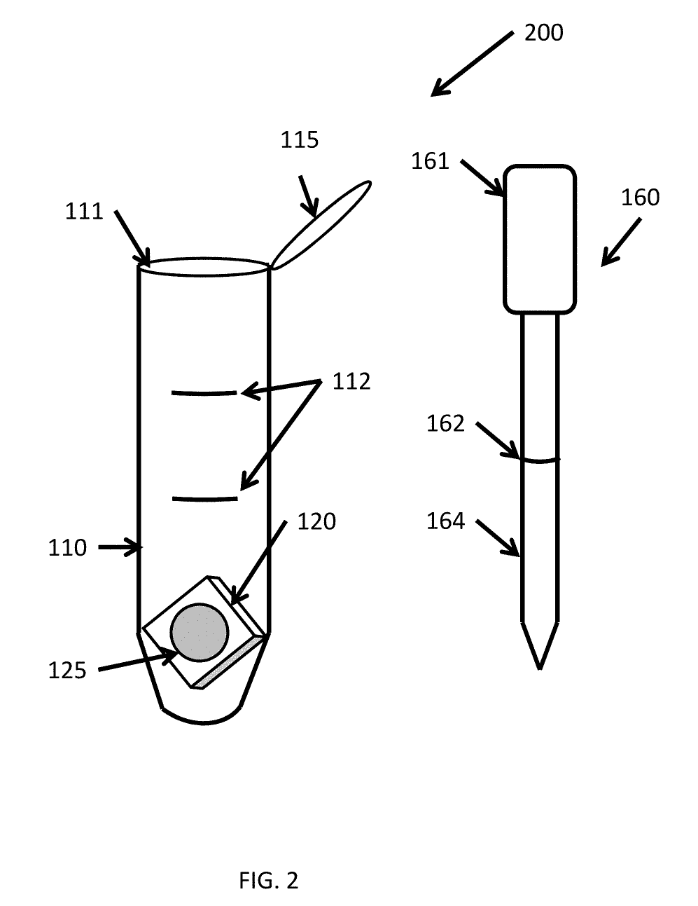

FIG. 2 illustrates a kit in accordance with various embodiments described herein. The kit can include a system 200 for measuring the concentration of an analyte and instructions. The system 200 can include a vial 110 having an open end and a conjugate pad 120 including dry probe material 125 and sized to be disposed within the vial 110. The vial 110 and conjugate pad 120 can be substantially as described above with relation to FIG. 1. The system can also include a pipette 160. By following the instructions included in the kit, a user can produce a sample in solution that is ready for analysis using, for example, a lateral flow device.

The pipette 160 may come in a variety of shapes and include a variety of materials. In some embodiments, the pipette 160 can be a one-piece disposable unit. The pipette 160 can be made of molded or blow-molded plastic in some embodiments. The pipette can include a bulb portion 161 and a stem portion 164. By compressing and decompressing the bulb portion 161, a user can draw liquid up into the stem portion 164 or expel the liquid from the stem portion 164, respectively. In other embodiments, the stem portion 164 can be made of glass and the bulb portion 161 can be made of rubber. The bulb portion 161 and the stem portion 164 can be shipped separately or attached in some embodiments. In some embodiments, the pipette 160 can be designed to transfer a fixed volume of liquid. For example, the pipette 160 can be an exact-volume transfer pipette.

The pipette 160 can include one or more volume indicator marks 162. The volume indicator mark 162 can indicate a location on the pipette 160 wherein liquid filled to that location will be at a specified volume to reconstitute a desired concentration of probe material in solution. In some embodiments, the one or more volume indicator marks 162 can indicate volumes of 200 μL, 500 μL, 750 μL, 1 mL, 2 mL, 5 mL, or any other suitable volume.

The kit can include instructions for performing a measurement of analyte concentration. The instructions can describe (a) drawing a liquid sample including the analyte into the pipette to one of the one or more fill indicator lines; (b) dispensing the liquid sample into the vial; (c) agitating the liquid in the vial to reconstitute the dry probe material; and (d) contacting a lateral flow test strip of a lateral flow device with the liquid sample in the vial.

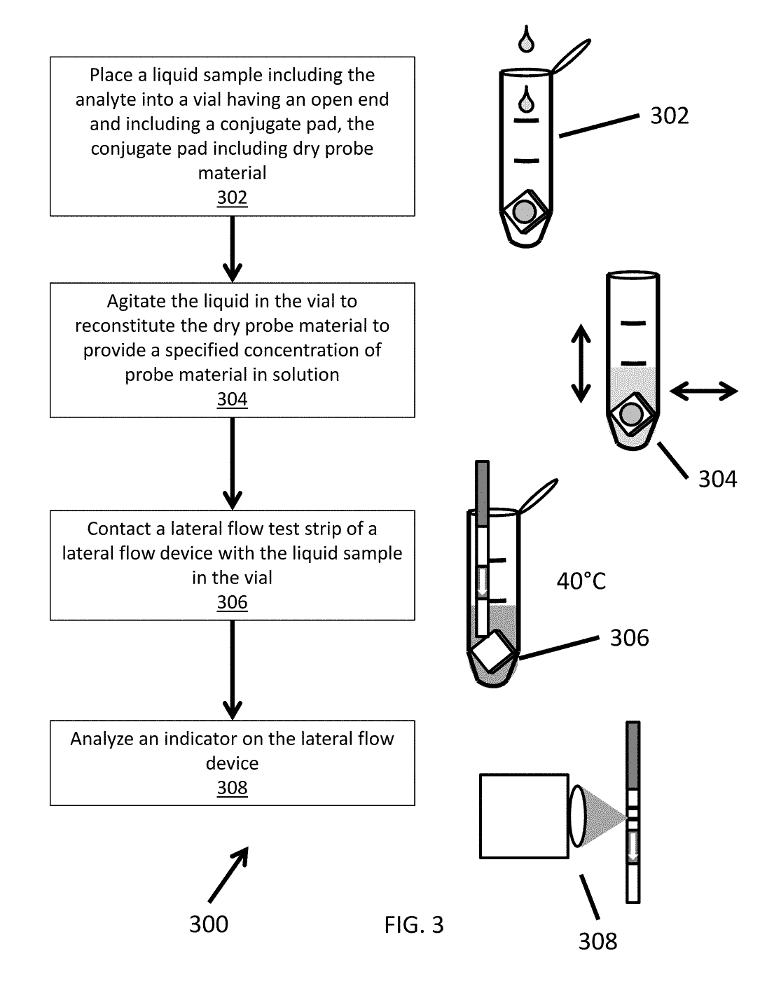

FIG. 3 illustrates a method 300 of measuring the concentration of an analyte in accordance with various embodiments described herein. In FIG. 3, each step of the method 300 is illustrated by an accompanying drawing to The method 300 includes placing a liquid sample including the analyte into a vial having an open end and including a conjugate pad (step 302). The conjugate pad includes dry probe material. In some embodiments, the vial 110 and conjugate pad 120 including dry probe material 125 can be as described above with reference to FIGS. 1 and 2. For example, a pipette can be used to place a specific volume of liquid sample into the vial.

The method 300 also includes agitating the liquid in the vial to reconstitute the dry probe material to provide a specified concentration of probe material in solution (step 304). For example, the vial can be shaken, stirred, or vortexed to agitate the liquid and cause the dry probe material to reconstitute at a specified concentration. The method 300 also includes contacting a lateral flow test strip of a lateral flow device with the liquid sample in the vial (step 306). In some embodiments, the lateral flow device 150 including lateral flow test strip 152 can be used as described above with reference to FIG. 1.

The method 300 also includes analyzing an indicator on the lateral flow device (step 308). In some embodiments, the indicator can be one of the control indicator line 154 or the test indicator line 155 described above with reference to FIG. 1. In some embodiments, a lateral flow reader such as the Vertu (commercially available from VICAM, Milford, Mass.) can be used to analyze the indicator on the lateral flow device. The lateral flow reader can illuminate the lateral flow device and measure the absorption or extinction of light caused by the indicator. The absorption or extinction can be indicative of a concentration of an analyte in solution.

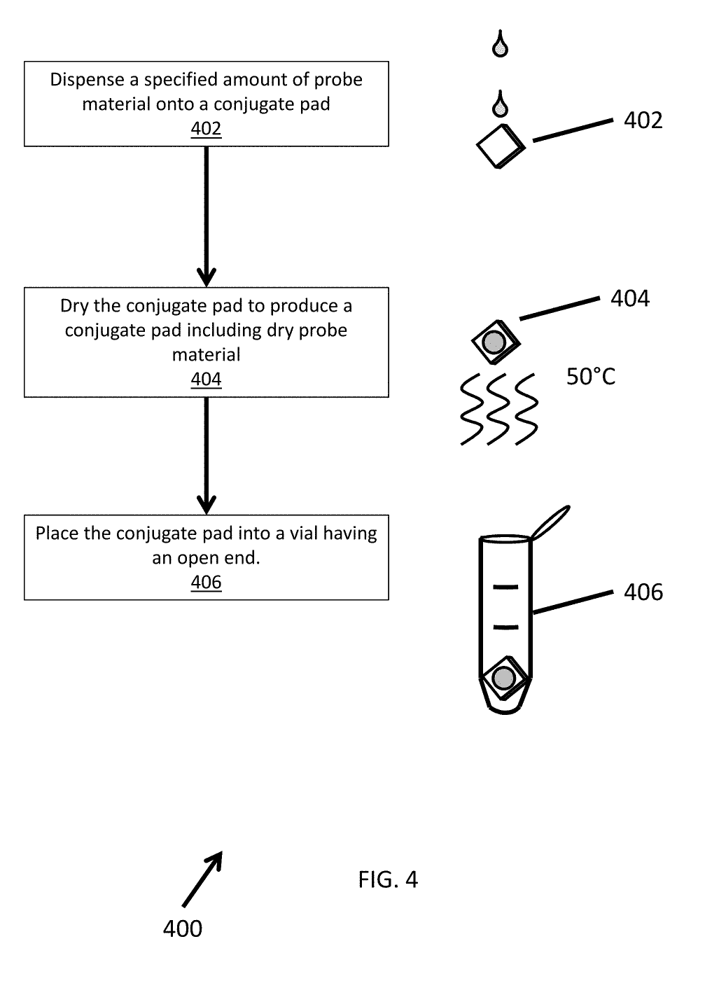

FIG. 4 illustrates a method 400 of producing an analyte measurement system in accordance with various embodiments described herein. The method 400 includes dispensing a specified amount of probe material onto a conjugate pad (step 402). For example, a liquid dispensing system can be used to dispense probe material onto the conjugate pad as described above with reference to FIG. 1. The method 400 also includes drying the conjugate pad to produce a conjugate pad including dry probe material (step 404). For example, the conjugate pad can be dried using forced hot air, radiant heat, or other appropriate mechanisms.

The method 400 also includes placing the conjugate pad into a vial having an open end (step 406). For example, the conjugate pad 120 can be placed into a vial 110 as described above with reference to FIG. 1. While the above method references a conjugate pad, it is noted that the pad does not have to be a single pad, but rather in some embodiments, the conjugate pad of steps 402 and 404 can be a strip or roll of pad material to create multiple pads for use in step 406.

Although systems and methods described above focus on measurement of an analyte concentration in solution, embodiments of the systems and methods described herein are not limited only to analyte measurements. The systems and methods described herein contemplate the ability to dry and reconstitute any sample in solution for a variety of experimental conditions as described in greater detail below.

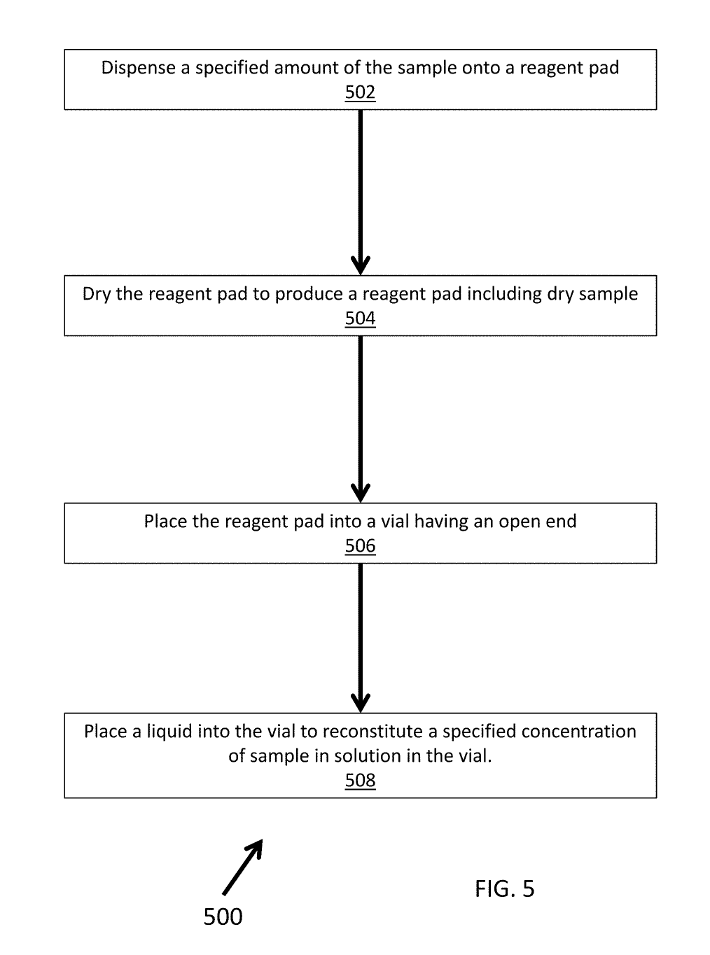

FIG. 5 illustrates a method 500 of producing a sample in solution in accordance with various embodiments herein. The method can advantageously be used to provide a specified concentration of a sample in solution for use in further experiments. Non-limiting examples of the use of reconstituted samples in accordance with embodiments described herein include providing prescribed concentrations or amounts of acid or base to adjust the pH of a solution; providing a prescribed concentration of enzymes in to digest material provided in solution (e.g., pepsin digestion); providing a prescribed concentration of surfactants to disrupt cell membranes and allow extraction of internal components of cells in solution; providing a prescribed concentration or amount of surfactants to extract hydrophobic analytes in solution; providing a prescribed concentration of inhibitors to suppress unwanted enzyme activity in a sample; or providing a fixed amount of analyte to be reconstituted and used as QC control samples.

The method for producing a sample in solution of the present disclosure provides several advantages over the prior art. In conventional systems, preparation of individual samples in solution can be time-consuming and require specialized equipment. In systems and methods described herein, the sample is prepared in bulk at low-cost and dried onto the reagent pad. The reagent pad can be non-reactive and configured to release the dried sample with high efficiency. Thus, preparing the sample using systems and methods described herein does not require specialized equipment or knowledge on the part of the end user.

The method 500 includes dispensing a specified amount of the sample onto a reagent pad (step 502). In some embodiments, the reagent pad can be substantially similar to the conjugate pad 120 as described above with reference to FIGS. 1 and 2. In some embodiments, the reagent pad can include modifications or properties that make the reagent pad amenable to drying the sample. For example, the reagent pad may be naturally or artificially hydrophobic to hydrophilic to more quickly absorb the sample depending upon the sample properties. The reagent pad in some embodiments may be particularly robust against degradation due to the sample. For example, the reagent pad may be resilient to acids or bases for use with acidic or basic samples, respectively. Dispensing the sample onto the reagent pad may be performed as described above with reference to the conjugate pad. For example, the reagent pad may be unspooled while liquid sample is dispensed onto the reagent pad at a controlled rate using a liquid dispensing system.

The method 500 also includes drying the reagent pad to produce a conjugate pad including dry sample (step 504). For example, the reagent pad can be dried using forced hot or room-temperature air, radiant heat, or other methods. The method 500 also includes placing the reagent pad into a vial having an open end (step 506). In some embodiments, a vial 110 having an open end may be used as described above in relation to FIG. 1

The method 500 also includes placing a liquid into the vial to reconstitute a specified concentration of sample in solution in the vial (step 508). In some embodiments, the liquid can include an analyte or other material including cells that is intended to interact with the reconstituted sample.

When an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements, device components or method steps, those elements, components or steps may be replaced with a single element, component, or step. Likewise, a single element, component, or step may be replaced with a plurality of elements, components, or steps that serve the same purpose. Moreover, while exemplary embodiments have been shown and described with references to particular embodiments thereof, those of ordinary skill in the art will understand that various substitutions and alterations in form and detail may be made therein without departing from the scope of the invention. Further still, other embodiments, functions, and advantages are also within the scope of the invention.

1. A system for measuring the concentration of an analyte, comprising:
a vial having an open end; and
a conjugate pad including dry probe material and sized to be disposed within the vial,
wherein the conjugate pad includes an amount of dry probe material such that placing the conjugate pad in contact with a liquid sample including the analyte within the vial reconstitutes the dry probe material to provide a specified concentration of probe material in solution.

2. The system of claim 1, wherein the dry probe material includes conjugated metal nanoparticles.

3. The system of claim 1, wherein the dry probe material includes polymer nanoparticles.

4. The system of claim 1, wherein a length and a width of the conjugate pad are each in a range from 2 mm to 20 mm.

5. The system of claim 1, further comprising a lateral flow device and wherein contacting a lateral flow test strip of the lateral flow device with the liquid sample in the vial causes an indicator to appear on the lateral flow device.

6. The system of claim 1, wherein the vial includes a volume indicator mark to indicate a volume of the liquid sample to be placed therein.

7. The system of claim 1, wherein the amount of probe material dispensed on the conjugate pad is in a range from 0.01 to 2 optical density measured at maximum absorption wavelength when reconstituted in the liquid sample.

8. A method of measuring the concentration of an analyte, comprising:
placing a liquid sample including the analyte into a vial having an open end and including a conjugate pad, the conjugate pad including dry probe material;
agitating the liquid in the vial to reconstitute the dry probe material to provide a specified concentration of probe material in solution;
contacting a lateral flow test strip of a lateral flow device with the liquid sample in the vial; and
analyzing an indicator on the lateral flow device.

9. The method of claim 8, wherein the dry probe material includes conjugated metal nanoparticles.

10. The method of claim 8, wherein the dry probe material includes conjugated polymer nanoparticles.

11. The method of claim 8, wherein analyzing the indicator on the lateral flow device includes:
obtaining an intensity of the indicator; and
comparing the intensity of the indicator to one or more reference intensities to determine a concentration of the analyte in the liquid sample.

12. The method of claim 8, wherein placing the liquid sample into the vial includes filling the vial until the level of the liquid sample reaches a volume indicator mark.

13. The method of claim 8, wherein a length and a width of the conjugate pad are each in a range from 2 mm to 20 mm.

14. The method of claim 8, wherein the amount of probe material dispensed on the conjugate pad is in a range from 0.01 to 2 optical density measured at maximum absorption wavelength when reconstituted in liquid sample.

15.-21. (canceled)

22. A kit comprising
(i) a system for measuring the concentration of an analyte, comprising:
(a) a vial having an open end; and
(b) a conjugate pad including dry probe material and sized to be disposed within the vial, wherein the conjugate pad includes an amount of dry probe material such that placing the conjugate pad in contact with a liquid sample including the analyte within the vial reconstitutes the dry probe material to provide a specified concentration of probe material in solution; and
(c) a pipette including one or more fill indicator lines to indicate volumes of fluid for reconstituting the dry probe material to a specified concentration, and
(ii) instructions for
(a) drawing a liquid sample including the analyte into the pipette to one of the one or more fill indicator lines;
(b) dispensing the liquid sample into the vial;
(c) agitating the liquid in the vial to reconstitute the dry probe material; and
(d) contacting a lateral flow test strip of a lateral flow device with the liquid sample in the vial.
